(19) United States
(12) Patent Application Publication
(10) Pub. No.: US 2025/0241253 A1
(43) Pub. Date: Jul. 31, 2025

(54) METHOD FOR OBTAINING NEW DISTANT GERMPLASM OF INTERGENERIC HYBRIDIZED POTATO BASED ON A GRAFTING-MENTOR

(71) Applicant: Hebei North University, Zhangjiakou (CN)

(72) Inventors: Lipan QI, Zhangjiakou (CN); Kuan WANG, Zhangjiakou (CN); Yan FENG, Zhangjiakou (CN); Yating LUO, Zhangjiakou (CN); Lei WANG, Zhangjiakou (CN); Guili WU, Zhangjiakou (CN); Yue LI, Zhangjiakou (CN); Jiang YIN, Zhangjiakou (CN); Yan WANG, Zhangjiakou (CN); Chang LIU, Zhangjiakou (CN)

(21) Appl. No.: 19/034,276

(22) Filed: Jan. 22, 2025

(30) Foreign Application Priority Data
Dec. 21, 2023 (CN) 202311773374.2

(51) Int. Cl. A01H 1/02; A01G 2/35; A01G 22/25

(52) U.S. Cl. CPC A01H 1/021; A01G 2/35; A01G 22/25

(57) ABSTRACT

A method is provided for obtaining new distant germplasm of intergeneric hybridized potato based on grafting-mentor, including the following steps: (1) using young branches, which are growing well, free of diseases, non-lignified, and from an upper part of Lycium as rootstock; (2) using seedlings of diploid primitive cultivated potato with 2-3 unfolded leaves as scion; (3) grafting; (4) conducting post-grafting management; (5) obtaining distant hybridized germplasm by using potato flower buds from the grafted plants for pollination, obtaining seeds of potato varieties after self-fertility, performing pre-germination on selfing seeds, obtaining true seed mini-tubers, and using these mini-tubers for further grafting and pollination to obtain distant hybridized seeds, performing tissue culture propagation on the distant hybridized seeds to obtain F1 asexual clone line foundation seeds.

### CROSS-REFERENCE TO RELATED DISCLOSURES

This application claims priority to Chinese Patent Application No. 202311773374.2, filed on Dec. 21, 2023, entitled “A Method for Obtaining New Distant Germplasm of Intergeneric Hybridized Potato Based on Grafting-Mentor”, which is hereby incorporated by reference in its entirety.

### TECHNICAL FIELD

The present disclosure relates to a technical field of potato breeding technology, specifically to a method for obtaining new distant germplasm of intergeneric hybridized potato based on a grafting-mentor.

### BACKGROUND

Potatoes originated in the Andes of South America. Globally, the genetic base of cultivated species of potato is narrow, the potato experiences severe inbreeding depression and resistance breeding is limited by the resources. Lycium, as a member of the Solanaceae family like potato, is resistant to late blight, drought, and salinity, so that Lycium is regarded as a pioneer species for afforestation in saline-alkali soils. Grafting, a technique commonly used in agricultural and forestry production, plays a significant role in improving production efficiency, enhancing quality, preserving germplasm resources, introducing new mutant genes, and so on. Currently, late blight is one of the most significant diseases threatening potato production, and causing substantial losses to the industry every year. There are studies showing that genomic exchanges may occur between rootstocks and scions during grafting, and vascular bundles and plasmodesmata may provide conditions for horizontal gene transfer. Stegemann and Rock conduct reciprocal grafting by using two transgenic tobacco lines, and nuclear and plastid genes carried different markers and reporter genes. The detection of reporter genes demonstrated short-distance transfer of plastid genomes at the grafting interface. If disease resistance genes from Lycium could be transferred to potato plants via grafting, so as to obtain new germplasm resources or intermediate materials, new insights would be provided for researches on late blight resistance of potato. Ohta (1991) from Japan conducted years of experiments on grafting hybridization for peppers, and not only obtained graft hybrids, but also further studied them at a genetic level. According to the study by Ohta, some genetic variation on Mendelian characters after grafting has been found and it is further discovered that a mechanism by which these variations are induced is similar to transformation mechanism in higher organisms. Stegemann and Bock (2006) from Germany grafted two transgenic tobacco lines, and found that marker genes could frequently exchange between cells at the grafting site, which indicates that horizontal gene transfer occurs between rootstock and scion.

Currently, there are no domestic or international reports on overcoming the barriers in sexual hybridization between distant plants of potatoes by using grafting mentor with potato as maternal parent and Lycium as male parent to obtain intergeneric hybridized progeny.

### BRIEF SUMMARY

The technical problem addressed by the present disclosure is to provide a method for obtaining new distant germplasm of intergeneric hybridized potato based on a grafting-mentor, to overcome the barriers in sexual hybridization between distant plants of potatoes and obtain intergeneric hybridized progeny.

One of the technical problems addressed by the present disclosure may be implemented with the following technical solution:

A method for obtaining new distant germplasm of intergeneric hybridized potato based on a grafting-mentor, including the following steps:
(1) cultivating and selecting a rootstock using young branches, which are growing well, free of diseases, non-lignified, and from an upper part of Lycium as rootstock;
(2) cultivating and selecting a scion using seedlings of diploid primitive cultivated potato with 2-3 unfolded leaves as scion;
(3) grafting by cutting the seedlings of potato at base with a blade, removing all leaves thereof, and trimming the scion into a wedge shape with two slopes, which are symmetrical to each other and have a size matched with a size of a cut of the rootstock, performing cleft grafting with the surfaces of the wedge being clean and smooth by inserting the scion into the cut of the rootstock with the scion and the rootstock aligned with each other at the cut, and then securing the scion and the rootstock together with plastic film;
(4) performing conducting management on the grafted plants after grafting;
(5) obtaining distant hybridized germplasm by using potato flower buds from the grafted plants for pollination, obtaining seeds of potato varieties after self-fertility, performing pre-germination on selfing seeds, obtaining true seed mini-tubers, and using these mini-tubers for further grafting and pollination to obtain distant hybridized seeds, performing tissue culture propagation on the distant hybridized seeds to obtain F1 asexual clone line foundation seeds.

Preferably, the step (5) includes the following steps:
(5-1) removing stamens of potato flower buds from grafted seedling that are plump and in a state to be blooming in about 1-2 days upon pushing petals of the potato flower buds aside;
(5-2) performing pollination, bagging, and tagging on the potato flower buds with the stamens removed, wherein the pollination includes collecting Lycium pollen and pollinating potato/Lycium grafted seedlings every morning;
(5-3) performing ovary enlargement including delaying abscission time of the grafted plant by 5-7 days compared to control groups wherein ovary of first generation grafted potato rarely enlarges after pollination with Lycium pollen, and collecting selfing seeds of potato which has good compatibility in distant hybrid and vigorous growth in scion, and has the capability of self-fertility;
(5-4) performing pre-germination on selfing seeds, and obtaining true seed mini-tubers including planting seeds obtained by pre-processing the hybridized seeds from scion obtained in previous year and mixing the pre-processed seeds with find sand, to obtain true seed mini-tubers in autumn;
(5-5) performing grafting on the true seed mini-tubers including planting the true seed mini-tubers obtained in step (5-4), and grafting seedlings obtained during a seedling emergence after the planting of the true seed mini-tubers, which are progeny from the same parent of the true seed mini-tuber, and referred as sibling lines, with Lycium in a same way as in step (3);
(5-6) performing pollination on scions of the sibling lines including collecting Lycium pollen and pollinating potato/Lycium grafted seedlings every morning;
(5-7) performing ovary enlargement of the scions of the sibling lines wherein the ovary enlarges in 7-10 days after pollination;
(5-8) performing aseptic culture of distant hybridized seeds including planting the distant hybridized seeds on ½ MS medium, which may be performed in a tissue culture room, performing tissue propagation on planted distant hybridized seeds, and planting stem segments obtained by asexually propagation in greenhouse vermiculite to obtain F1 asexual clone line foundation seeds.

Preferably, the way for performing pollination in steps (5-2) and (5-6) involves collecting pollen of male parent Lycium, applying the collected pollen onto stigma of emasculated flowers, bagging the stigma applied with pollen with labels marking hybridization date and the names of the parent varieties.

Preferably, step (5-4) specifically includes: soaking hybridized seeds of scions obtained in the previous year in 1,000 times gibberellin solution for 48 hours; mixing the seeds with fine sand, spreading the seeds mixed with sand in seed trays evenly to cover the seeds with a layer of soil; transplanting seedlings into 10 cm×10 cm pots in 40 days after planting to perform standard management of water and fertilizer, and obtaining true seed mini-tubers in autumn.

Preferably, step (5-5) specifically includes: planting a series of true seed mini-tubers, which are obtained from self-fertility seeds of same variety and referred as sibling lines, after grafting potato with Lycium, and grafting seedlings from the planted sibling lines in a same way as in step (3).

Preferably, the rootstock used in step (1) is processed as follows: removing top bud of Lycium and cutting the Lycium at internode between first and second fully developed leaves from top of the rootstock vertically by using a double-edged blade to a depth of 1.0-1.5 cm.

Preferably, the step (3) further includes sterilizing the blade and hands with 75% alcohol before grafting; disinfecting the blade and hands after each grafting with the alcohol, and waiting until the alcohol evaporates completely before proceeding to the next step.

The above technical solution of the present disclosure has the following beneficial effects:

The present disclosure is proposed in view of that the genetic base of cultivated species of potato is narrow globally, the potato experiences severe inbreeding depression and resistance breeding is limited by the resources, while late blight is one of the most significant diseases threatening potato production currently. The present disclosure is proposed further in consideration that Lycium is resistant to late blight, drought, and salinity, and is a member of the Solanaceae family like potato. The technical solution provided in the present disclosure uses Lycium and potato as basis and effectively reduces the incompatibility between Lycium and potato, rapidly achieves distant grafting of Lycium and potato, and obtains distant hybridized progeny for the first time, so that a material foundation is provided for broadening potato resistance breeding resources and distant sexual hybridization.

### BRIEF DESCRIPTION OF THE DRAWINGS

FIGS. 1(A) to 1(H) show morphological characteristics of a grafted plant obtained by grafting potato with Lycium, in which, FIG. 1(A) shows external condition;

FIG. 1(B) shows initial stage; FIG. 1(C) shows healing; FIG. 1(D) shows modified stem of scion; FIG. 1(E) shows: growing stage; FIG. 1(F) shows flowering stage; FIG. 1(G) shows ovary enlargement after pollination with Lycium pollen; FIG. 1(H) shows self-fertility of scion during maturity stage.

FIG. 2(A), FIG. 2(B), and FIG. 2(C) show views of the ovary enlargement in 20 days after pollination on potato/Lycium grafted plants with Lycium pollen.

FIG. 3 shows the progeny of potato/Lycium grafted plants obtained in the Lycium ovary.

FIGS. 4(A) and 4(B) show the production of mini-tubers, wherein FIG. 4(A) shows seedlings-in-bottle propagated through tissue culture are transplanted into a vermiculite substrate with coconut coir; FIG. 4(B) shows F1 mini-tubers are obtained.

FIG. 5 is a schematic view showing a flowchart of the method for obtaining a new distant germplasm of intergeneric hybridized potato based on a grafting-mentor provided by embodiments of the present disclosure.

### DETAILED DESCRIPTION

Detailed description is made on embodiments of the present disclosure below for better understanding of the present disclosure.

Unless otherwise stated, all experimental methods used in the following embodiments are conventional methods. Materials, reagents, etc., used in the following embodiments are commercially available unless otherwise stated.

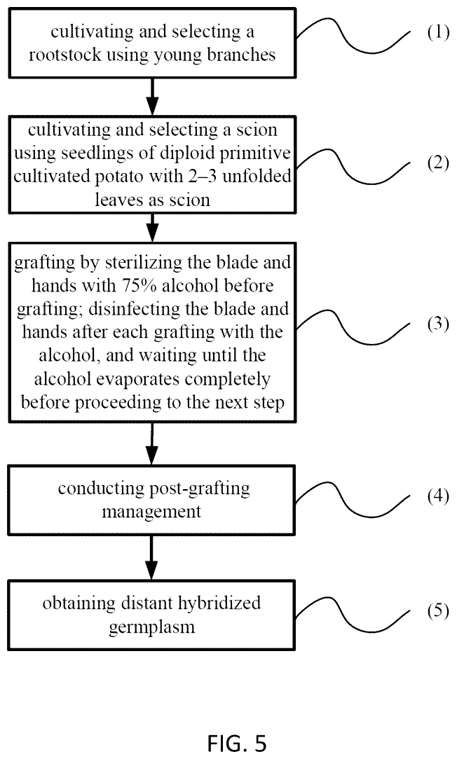

FIG. 5 is a schematic view showing a flowchart of the method for obtaining a new distant germplasm of intergeneric hybridized potato based on a grafting-mentor provided by embodiments of the present disclosure. As shown in FIG. 5, the method for obtaining a new distant germplasm of intergeneric hybridized potato based on a grafting-mentor provided by embodiments of the present disclosure, including the following steps:
(1) cultivating and selecting a rootstock using young branches, which are growing well, free of diseases, non-lignified, and from an upper part of Lycium as rootstock.
(2) cultivating and selecting a scion using seedlings of diploid primitive cultivated potato with 2-3 unfolded leaves as scion.
(3) grafting by sterilizing the blade and hands with 75% alcohol before grafting; disinfecting the blade and hands after each grafting with the alcohol, and waiting until the alcohol evaporates completely before proceeding to the next step. More particularly, the step (3) includes the following steps:
(3-1) preparing rootstock including removing top bud of Lycium and cutting the Lycium at internode between first and second fully developed leaves from top of the rootstock vertically by using a double-edged blade to a depth of 1.0-1.5 cm.
(3-2) determining variety of scion by using potato varieties with high graft compatibility with Lycium. The growth vigor of grafted plant of potato grafted with Lycium is significantly weaker than that of non-grafted potatoes. A height of scion plants after grafting is significantly lower than that of the control group (Table 1), with a reduction of 47.1%-52.8%. Although there is no significant difference in the number of stem nodes between grafted and control plants, the average internode length of the grafted plants is significantly shorter than that of the control plants, with an average reduction of 49.7% in the grafted group. The average number of branches in the group of grafted plant also decreases significantly.

TABLE 1
Comparison of Growth Vigor During the Flowering Stage Between
Grafted and Non-Grafted Potato of Different Varieties

| | | | | Average | |
|---|---|---|---|---|---|
| Potato | | Plant Height | Number of | internode | Number of |
| Variety | processing | (cm) | Stem Nodes | Length (mm) | Branches |
| J1703 | grafted | 33.81 ± 2.58 b | 11.11 ± 1.06a | 27.13 ± 2.33b | 3.41 ± 0.15b |
| | control | 68.61 ± 5.64 a | 11.50 ± 0.95a | 59.20 ± 3.56a | 4.93 ± 0.26a |
| J1701 | grafted | 72.10 ± 4.75b | 14.70 ± 1.12a | 39.15 ± 3.12b | 3.16 ± 0.50b |
| | control | 98.52 ± 8.62a | 14.61 ± 2.11a | 70.31 ± 4.20a | 4.51 ± 0.22a |
| J1702 | grafted | 40.71 ± 3.28b | 13.12 ± 1.39a | 33.11 ± 2.76b | 1.32 ± 0.61b |
| | control | 86.21 ± 6.21a | 13.73 ± 1.46a | 68.10 ± 3.89a | 4.22 ± 0.85a |
| Average | grafted | 48.87 ± 10.56b | 13.01 ± 2.35a | 33.10 ± 6.10b | 2.63 ± 1.14b |
| | control | 84.42 ± 15.29a | 13.31 ± 1.68a | 65.96 ± 5.96a | 4.52 ± 0.36a |

Note:
Different lowercase letters in the same column indicate significant differences at a level of 0.05 in multiple-range tests. Values in the table are expressed as mean ± standard error.

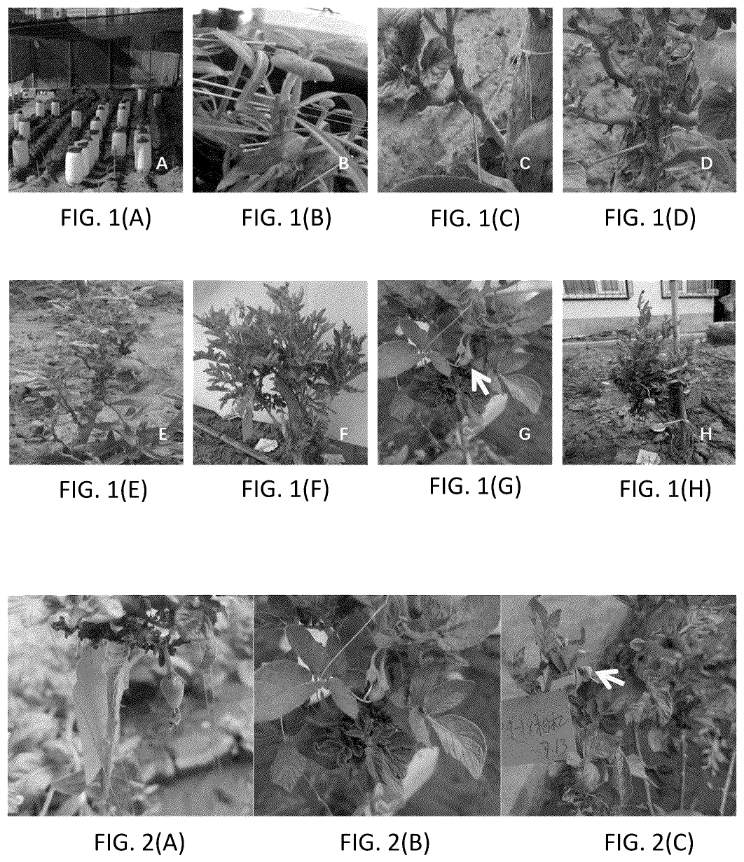

(3-3) grafting by cutting the seedlings at base with a blade, removing all leaves thereof, and trimming the scion into a wedge shape with two slopes, which are symmetrical to each other and have a size matched with a size of a cut of the rootstock, performing cleft grafting with the surfaces of the wedge clean and smooth by inserting the scion into the cut of the rootstock with the scion and the rootstock aligned with each other at the cut, and then securing the scion and the rootstock together with plastic film;
(4) conducting post-grafting management including:
covering the area where the grafting is performed with a plastic bag after grafting, and performing culturing under constant warmth and humidity (see FIG. 1(A) and FIG. 1(B)), spraying Carbendazim at 1 g/L on the grafted plant daily to prevent bacterial growth;
exposing the grafted plant under sun 7 days later (after 7 days after grafting), and gradually extending the exposing time as the part where the grafting is performed grows;
completely removing measures for shading and keeping warm when the scion of the grafted potato has 2-3 leaves grown 20-25 later (after 20-25 days), and conduct standard management on the grafted plant.

It should be noted that, new shoots from the Lycium rootstock at the part where grafting is performed needs to be removed. Furthermore, the grafted plant may be irrigated with total nutrient solution and sprayed with fungicides and insecticides regularly to provide sufficient nutrition and prevent pest and diseases.

The grafted potato with Lycium may survive and enter a vigorous growth stage 10-15 days after grafting. Stolons of the scion may fail to form tubers in the soil, so that the nutrients may fail to be transported into storage organs (underground tubers), and thus the stem at the area where the grafting is performed may enlarge, and some of the stolons may grow into shoots (see FIG. 1(D)).

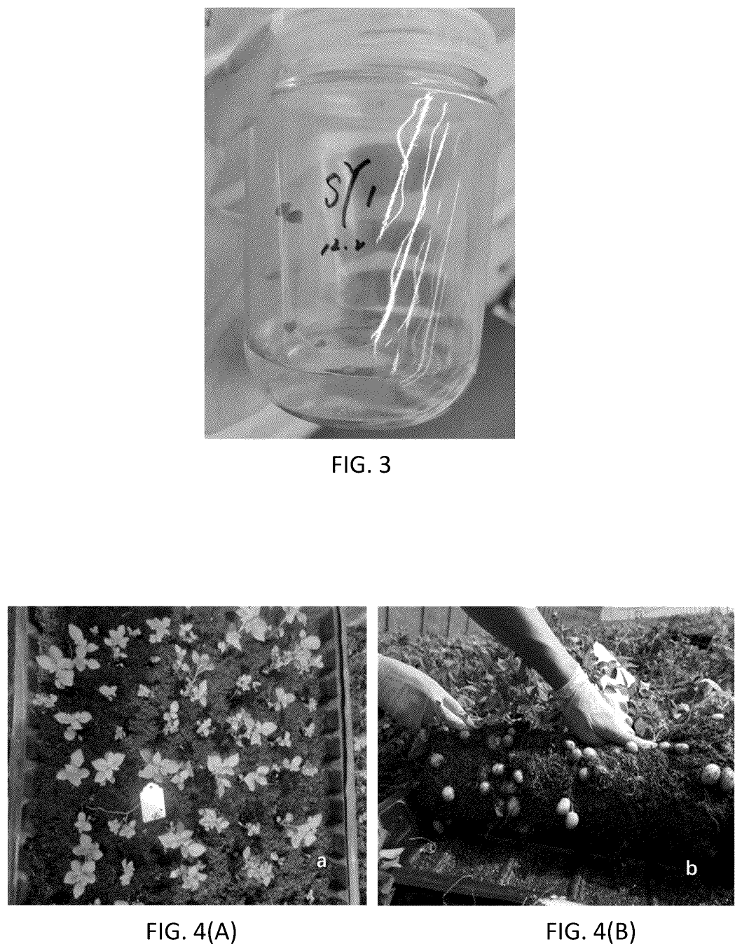

The scion may grow well with internodes shortened and growth vigor weakened (see FIG. 1(E)).
(5) obtaining distant hybridized germplasm including:
(5-1) removing stamens of potato flower buds from grafted seedling that are plump and in a state to be blooming in about 1-2 days upon pushing petals of the potato flower buds aside, the scion of grafted potato with strong compatibility may bloom normally at late growth stage (see FIG. 1(F));
(5-2) performing pollination, bagging, and tagging on the potato flower buds with the stamens removed, wherein the pollination includes collecting Lycium pollen and pollinating potato/Lycium grafted seedlings every morning; collecting pollen of male parent Lycium, applying the collected pollen onto stigma of emasculated flowers, bagging the stigma applied with pollen with labels marking hybridization date and the names of the parent varieties;
(5-3) performing ovary enlargement including delaying abscission time of the grafted plant by 5-7 days compared to control groups wherein ovary of first generation grafted potato rarely enlarges after pollination with Lycium pollen, and collecting seeds of self-fertile potato which has good compatibility in distant hybrid and vigorous growth in scion, and has the capability of self-fertility (see FIG. 1(H));
(5-4) performing pre-germination on selfing seeds, and obtaining true seed mini-tubers including soaking hybridized seeds of scions obtained in the previous year in 1,000 times gibberellin solution for 48 hours; mixing the seeds with fine sand, spreading the seeds mixed with sand in seed trays evenly to cover the seeds with a layer of soil; transplanting seedlings into 10 cm×10 cm pots in 40 days after planting to perform standard management of water and fertilizer, and obtaining true seed mini-tubers in autumn;
(5-5) performing grafting on the true seed mini-tubers including planting the true seed mini-tubers obtained in step (5-4), and grafting seedlings obtained during a seedling emergence after the planting of the true seed mini-tubers, which are progeny from the same parent of the true seed mini-tuber, and referred as sibling lines, with Lycium in a same way as in step (3);
(5-6) performing pollination on scions of the sibling lines including collecting Lycium pollen and pollinating potato/Lycium grafted seedlings every morning; collecting pollen of male parent Lycium, applying the collected pollen onto stigma of emasculated flowers, bagging the stigma applied with pollen with labels marking hybridization date and the names of the parent varieties;
(5-7) performing ovary enlargement of the scions of the sibling lines, wherein the ovary enlarges in 7-10 days after pollination (see FIGS. 2(A), 2(B), and 2(C));
(5-8) performing aseptic culture of distant hybridized seeds including planting the distant hybridized seeds on ½ MS medium in a tissue culture room (see FIG. 3), performing tissue propagation on planted distant hybridized seeds, and planting stem segments obtained by asexually propagation in greenhouse vermiculite to obtain F1 asexual clone line foundation seeds (see FIGS. 4(A) and 4(B)).

Although the present disclosure has been disclosed with reference to the above embodiments, description is not intended to limit the present disclosure. Various modifications and alternatives can be made by those skilled in the art without departing from the spirit and scope of the present disclosure. Therefore, the scope of the present disclosure is defined by the claims and their equivalents.

1. A method for obtaining a new distant germplasm of intergeneric hybridized potato based on a grafting-mentor, comprising the following steps:
(1) cultivating and selecting a rootstock using young branches, which are growing well, free of diseases, non-lignified, and from an upper part of Lycium as rootstock;
(2) cultivating and selecting a scion using seedlings of diploid primitive cultivated potato with 2-3 unfolded leaves as scion;
(3) grafting by cutting the seedlings of potato at base with a blade, removing leaves thereof, and trimming the scion into a wedge shape with two slopes, which are symmetrical to each other and have a size matched with a size of a cut of the rootstock, performing cleft grafting with the surfaces of the wedge being clean and smooth by inserting the scion into the cut of the rootstock with the scion and the rootstock aligned with each other at the cut, and then securing the scion and the rootstock together with plastic film;
(4) conducting post-grafting management on the grafted plants after grafting;
(5) obtaining distant hybridized germplasm by using potato flower buds from the grafted plants for pollination, obtaining seeds of potato varieties after self-fertility, performing pre-germination on selfing seeds, obtaining true seed mini-tubers, and using these mini-tubers for further grafting and pollination to obtain distant hybridized seeds, performing tissue culture propagation on the distant hybridized seeds to obtain F1 asexual clone line foundation seeds.

2. The method for obtaining a new distant germplasm of intergeneric hybridized potato based on a grafting-mentor according to claim 1, wherein the step (5) comprises the following steps:
(5-1) removing stamens of potato flower buds from grafted seedling that are plump and in a state to be blooming in about 1-2 days upon pushing petals of the potato flower buds aside;
(5-2) performing pollination, bagging, and tagging on the potato flower buds with the stamens removed, wherein the pollination comprises: collecting Lycium pollen and pollinating potato/Lycium grafted seedlings every morning;
(5-3) performing ovary enlargement comprising delaying abscission time of the grafted plant by 5-7 days compared to control groups wherein ovary of first generation grafted potato rarely enlarges after pollination with Lycium pollen, and collecting selfing seeds of potato which has good compatibility in distant hybrid and vigorous growth in scion, and has the capability of self-fertility;
(5-4) performing pre-germination on selfing seeds, and obtaining true seed mini-tubers comprising planting seeds obtained by pre-processing the hybridized seeds from scion obtained in previous year and mixing the pre-processed seeds with find sand, to obtain true seed mini-tubers in autumn;
(5-5) performing grafting on the true seed mini-tubers comprising planting the true seed mini-tubers obtained in step (5-4), and grafting seedlings obtained during a seedling emergence after the planting of the true seed mini-tubers, which are progeny from the same parent of the true seed mini-tuber, and referred as sibling lines, with Lycium in a same way as in step (3);
(5-6) performing pollination on scions of the sibling lines comprising collecting Lycium pollen and pollinating potato/Lycium grafted seedlings every morning;
(5-7) performing ovary enlargement of the scions of the sibling lines wherein the ovary enlarges in 7-10 days after pollination;
(5-8) performing aseptic culture of distant hybridized seeds comprising planting the distant hybridized seeds on ½ MS medium, performing tissue propagation on planted distant hybridized seeds, and planting stem segments obtained by asexually propagation in greenhouse vermiculite to obtain F1 asexual clone line foundation seeds.

3. The method for obtaining a new distant germplasm of intergeneric hybridized potato based on a grafting-mentor according to claim 2, wherein, the way for performing pollination in steps (5-2) and (5-6) comprises: collecting pollen of male parent Lycium, applying the collected pollen onto stigma of emasculated flowers, bagging the stigma applied with pollen with labels marking hybridization date and the names of the parent varieties.

4. The method for obtaining a new distant germplasm of intergeneric hybridized potato based on a grafting-mentor according to claim 2, wherein, the step (5-4) comprises:
soaking hybridized seeds of scions obtained in the previous year in 1,000 times gibberellin solution for 48 hours;
mixing the seeds with fine sand, spreading the seeds mixed with sand in seed trays evenly to cover the seeds with a layer of soil;
transplanting seedlings into 10 cm×10 cm pots in 40 days after planting to perform standard management of water and fertilizer, and obtaining true seed mini-tubers in autumn.

5. The method for obtaining a new distant germplasm of intergeneric hybridized potato based on a grafting-mentor according to claim 2, wherein the step (5-5) comprises: planting a series of true seed mini-tubers, which are obtained from self-fertility seeds of same variety and referred as sibling lines, after grafting potato with Lycium, and grafting seedlings from the planted sibling lines in a same way as in step (3).

6. The method for obtaining a new distant germplasm of intergeneric hybridized potato based on a grafting-mentor according to claim 1, wherein the rootstock used in step (1) is processed as follows: removing top bud of Lycium and cutting the Lycium at internode between first and second fully developed leaves from top of the rootstock vertically by using a double-edged blade to a depth of 1.0-1.5 cm.

7. The method for obtaining a new distant germplasm of intergeneric hybridized potato based on a grafting-mentor according to claim 1, wherein, the step (3) comprises: sterilizing the blade and hands with 75% alcohol before grafting; disinfecting the blade and hands after each grafting with the alcohol; and waiting until the alcohol evaporates completely before proceeding to the next step.
